(19) United States
(12) Patent Application Publication
(10) Pub. No.: US 2026/0014342 A1
(43) Pub. Date: Jan. 15, 2026

(54) NO SUPPLY INSTALLATION COMPRISING AN NO DELIVERY APPARATUS AND AN ANAESTHESIA VENTILATOR

(71) Applicant: INOSYSTEMS, Antony (FR)

(72) Inventors: Nathan PROUVEZ, Antony (FR); Yann BLANDIN, Antony (FR); Patricia THERRIAULT, Antony (FR); Juan MARTINEZ LAFRAYA, Antony (FR)

(21) Appl. No.: 19/242,507

(22) Filed: Jun. 18, 2025

(30) Foreign Application Priority Data
Jul. 9, 2024 (FR) 2407466

(51) Int. Cl. A61M 16/12; A61M 16/00; A61M 16/08; A61M 16/10

(52) U.S. Cl. CPC A61M 16/12; A61M 16/0833; A61M 16/085; A61M 16/1005; A61M 2016/0039; A61M 2202/0275

(57) ABSTRACT

The invention relates to an installation (100) for supplying a gas mixture containing NO to a patient, comprising an NO delivery apparatus (1) and a medical ventilator (50), an NO injection device (24), a flow rate measurement device (25) and a gas sampling module (61). The medical ventilator (50) is an anaesthesia ventilator (150) comprising a gas outlet (151) to which the flow rate measurement device (25) is connected. The NO injection device (24) is arranged downstream of the flow rate measurement device (25), and the gas sampling module (61) is arranged downstream of the NO injection device (24), at a distance (D) of at least 30 cm.

### TECHNICAL FIELD

The invention relates to an installation for supplying an NO-based gas mixture to a patient, comprising an NO delivery apparatus for supplying an NO-containing gas mixture, typically an NO/N₂ mixture, from one or more NO sources, such as pressurized gas cylinders, and an anaesthesia ventilator supplying an oxygen-based (i.e. >20 vol % approximately) gas.

### BACKGROUND

Inhaled nitric oxide (NO or iNO) is a gaseous medicament commonly used to treat patients suffering from acute pulmonary arterial hypertension, in particular pulmonary vasoconstriction in adults or children, including newborns (PPHN), as described for example in EP-A-560928 or EP-A-1516639.

In general, to implement therapy by inhaled NO, use is made of a gas supply installation, also known as an NO administration installation, comprising an NO delivery apparatus and a medical ventilator, that is to say a respiratory assistance apparatus, supplying a patient circuit.

The NO delivery apparatus makes it possible to inject a gas mixture based on NO, typically an NO/nitrogen mixture, into the patient circuit, which is also supplied with a gas flow containing oxygen (at least approximately 20 vol %), such as air or an oxygen/nitrogen mixture (O₂/N₂), supplied by the medical ventilator. This injection of NO is performed by means of an injection device or module arranged in the patient circuit.

The NO gas, typically an NO/nitrogen gas mixture, supplied to the NO delivery apparatus generally comes from one or more pressurized gas containers, such as gas cylinders.

The patient circuit generally comprises one or more flexible conduits which are fluidically connected to a respiratory interface, such as a tracheal intubation tube, a breathing mask or the like, which is used to supply to the patient to be treated a combined therapeutic gas mixture containing a given quantity or dose of NO, that is to say a dosage set by a doctor.

In order to regulate the quantity of NO supplied, use is usually made of a flow sensor arranged in the patient circuit, upstream of the NO injection site, which serves to measure the flow rate of gas containing oxygen supplied by the medical ventilator and to supply this flow rate measurement to the NO delivery apparatus.

Before being administered to the patient, the gas is usually humidified in a gas humidifier arranged in the patent circuit, between the respiratory interface and the flow sensor.

This type of installation is used in a hospital environment to administer the treatment by NO and thereby care for patients who need to inhale NO in order to treat their pulmonary arterial hypertension, notably during heart surgery. NO supply installations are described by EP3821929, EP4209243, EP4241817, EP4241812 and EP4295882.

However, when NO has to be administered to the patient during a surgical operation, for example during heart surgery, it may be necessary to use an anaesthesia ventilator, also referred to as an anaesthesia station, making it possible to also deliver an anaesthesia gas, such as isoflurane, desflurane or sevoflurane, used to sedate/anaesthetize the patient, during the surgery.

In this case, the patient circuit used is double-branched to bring the anaesthesia gas to the patient, via an inspiratory branch, then to recover, via an expiratory branch, the gases exhaled, which are rich in CO₂ and which still contain anaesthesia compounds.

The inspiratory and expiratory branches are generally connected at a junction piece, also referred to as a Y piece, which is also connected to the respiratory interface used to administer the gas to the patient and to recover the exhaled gases, such as a tracheal intubation tube or a breathing mask.

The NO delivery apparatus and the anaesthesia ventilator which is used instead of a conventional medical ventilator, must then be fluidically connected to the gas circuit used to administer the NO-based gas to the patient.

In particular, it is necessary to connect to the NO delivery apparatus not only the flow sensor arranged on the inspiratory branch which serves to measure the gas flow rate coming from the anaesthesia ventilator in order to allow regulation of the supply of NO (i.e. flow rate of NO) by the NO delivery apparatus, and the gas injector device serving to inject the NO into the inspiratory branch, but also the gas sampling line for taking gas samples from the NO-based gas mixture supplied to the patient in order to monitor the composition of this mixture and ensure that it contains the desired proportions of NO and oxygen and, conversely, does not contain excessive amounts of toxic species such as NO₂.

In an installation that uses a conventional ventilator, the NO injection module and the flow sensor are generally arranged in the inspiratory branch upstream of the gas humidifier and at a distance of at least 30 cm to 60 cm from the outlet of the ventilator, while the gas sampling line is usually connected upstream of the Y piece, typically at around 15 to 40 cm therefrom.

However, these connections pose a problem when the ventilator is not a conventional ventilator, but an anaesthesia ventilator.

To be specific, in this case, the interface with the NO delivery apparatus, in other words the connection of the gas sampling line and the flow sensor to the inspiratory branch, is not easy because installations that use anaesthesia ventilators generally use a more basic patient circuit, namely a simple flexible pipe or conduit without a gas humidifier because humidification takes place directly in the apparatus by means of on-board humidity and heat exchange filters, and with the Y piece sealed at the downstream outlet of this flexible pipe or conduit. Sometimes, the expiratory branch, which is generally also formed of a flexible pipe, maybe arranged coaxially in the flexible pipe acting as inspiratory branch.

Other known installations for supplying gas mixtures containing NO are notably described by US2023/270960, WO2016/096056 and WO2015/153713.

In all cases, it will be appreciated that such a basic patient circuit does not allow easy insertion of the gas sampling module supplying the gas sampling line, or the injection module and flow sensor, which must be connected to the NO delivery apparatus, without unsealing the Y piece in order to insert the gas sampling module and/or cutting the flexible pipe to insert therein the module comprising the flow sensor and the NO injector module, which inevitably impairs the patient circuit and/or the Y piece and, in all cases, is entirely inconceivable, for a surgical operation.

### SUMMARY

The invention aims to solve this problem of connecting the gas sampling module supplying the gas sampling line and the module comprising the flow sensor in a basic patient circuit associated with an anaesthesia ventilator, and in particular used in heart surgery.

A solution according to the invention thus relates to an installation for supplying a gas mixture containing NO, typically an NO/N₂ gas mixture, to a patient, i.e. a person, comprising:
an NO delivery apparatus configured to supply a flow of gas containing NO, and
a medical ventilator for supplying a flow of respiratory gas containing 02, to an inspiratory branch of a respiratory circuit,
an NO injection device, also referred to as an injection module, configured to inject the gas containing NO coming from the NO delivery apparatus into the flow of respiratory gas coming from the medical ventilator,
a flow rate measurement device configured to measure the flow rate of the flow of respiratory gas coming from the medical ventilator, and
a gas sampling module configured to sample, downstream of the NO injection device, some of the gas circulating in the inspiratory branch.

Furthermore, in the installation for supplying a gas mixture containing NO according to the invention, the medical ventilator is an anaesthesia ventilator comprising a gas outlet and the NO injection device is arranged downstream of the flow rate measurement device.

Moreover, in the installation for supplying a gas mixture containing NO according to the invention, the flow rate measurement device is connected directly to the gas outlet of the anaesthesia ventilator, and the gas sampling module is arranged downstream of the NO injection device and at a distance (D) of at least 30 cm from said NO injection device.

Depending on the embodiment concerned, the installation according to the invention may also comprise one or more of the following features:
the NO injection device is arranged between the flow rate measurement device and the gas sampling module.
the flow rate measurement device is fluidically connected to the NO injection device by means of at least one intermediate conduit portion.
the NO injection device is connected directly to the flow rate measurement device.
the injection device (or module) is configured to mix the gas containing NO coming from the NO delivery apparatus with the flow of respiratory gas containing O₂ supplied by the medical ventilator, and to obtain a combined gas mixture containing NO and oxygen, typically a combined gas mixture containing NO, oxygen and nitrogen.
several intermediate conduit portions fluidically connect the NO injection device to the gas sampling module.
the intermediate conduit portion or portions comprise pipe elements, preferably bendable or flexible, for example made of polymer.
it comprises tubular connectors for connecting the intermediate conduit portion or portions, preferably by fitting together.
tubular connectors fluidically connect said at least one intermediate conduit portion to the flow rate measurement device and to the NO injection device, and/or several intermediate conduit portions to one another.
the gas sampling module is arranged downstream of the injection device (or module) at a distance of between 30 cm and 80 cm, preferably at less than 60 cm, more preferably still between 30 and 50 cm.
the gas sampling module is fluidically connected (directly or indirectly) to the inspiratory branch of the respiratory circuit.
the gas sampling module is fluidically connected to an upstream end of the inspiratory branch of the respiratory circuit, in other words to the inlet of the inspiratory branch of a basic respiratory circuit, preferably a basic respiratory circuit comprising a junction piece (i.e. a Y piece) that is sealed (i.e. attached non-removably) at a downstream end of the inspiratory branch.
the NO injection device is fluidically connected to the NO delivery apparatus in such a way as to be supplied with gas containing NO by said NO delivery apparatus.
the flow rate measurement device is fluidically connected to the NO delivery apparatus in such a way as to supply it with flow rate measurements.
the flow rate measurement device comprises a flow sensor.
the flow sensor is a mass flow sensor.
the flow sensor is electrically connected to the NO delivery apparatus, in particular to the control means of the NO delivery apparatus.
the gas sampling module is fluidically connected to the NO delivery apparatus in such a way as to supply it with gas circulating in the inspiratory branch, downstream of the NO injection device.
the respiratory circuit further comprises an expiratory branch.
the inspiratory branch and the expiratory branch are fluidically connected at a joining piece, typically a Y piece.
the joining piece is fluidically connected to a respiratory interface, such as a breathing mask, an endotracheal intubation tube or the like.
the expiratory branch is fluidically connected to a gas inlet of the anaesthesia ventilator.
the respiratory circuit comprises flexible pipes or conduits, in particular the inspiratory branch and the expiratory branch.
it further comprises at least one NO container containing an NO/N₂ mixture, typically one or more pressurized gas cylinders.
the NO container or containers supply the NO delivery apparatus with a flow of gas containing NO, typically a gas mixture formed of nitrogen and NO, i.e. an NO/N₂ mixture.
the injection device comprises a first gas inlet supplied with a flow of respiratory gas containing 02, i.e. coming from the ventilator.
the injection device also comprises a second gas inlet supplied with gas containing NO coming from the NO delivery apparatus.
the injection device also comprises a gas outlet supplying the combined gas mixture containing NO and oxygen, obtained by mixing, within the injection device, the gas containing NO (e.g. NO/N₂ mixture) with the flow of respiratory gas containing O₂ (e.g. air or O₂/N₂ mixture).
the injection device or module comprises a module body comprising an internal gas passage (i.e. an internal volume or chamber) fluidically connected to the first gas inlet, to the second gas inlet and to the gas outlet.
the respiratory gas coming from the ventilator enters the internal gas passage of the injection device or module via the first gas inlet.
the NO-containing gas coming from the NO delivery apparatus enters the internal gas passage of the injection device or module via the second gas inlet.
the combined gas containing NO and oxygen leaves the injection device or module via the gas outlet.
the respiratory circuit, in particular the inspiratory branch, does not comprise any gas humidifier, i.e. it has no gas humidifier.
the flow rate measurement device, the NO injection device, the gas sampling module, said at least one intermediate conduit portion and the tubular connectors form an independent and removable connection assembly for connecting an NO delivery apparatus, configured to be fluidically connected between the inspiratory branch of the patient circuit and the gas outlet of the anaesthesia ventilator.

Moreover, depending on the embodiment in question, the NO delivery apparatus may comprise one or more of the following features:
it is supplied with a gas mixture formed of nitrogen and NO.
it comprises dose adjustment means which are configured to allow a user to fix or select a set NO content corresponding to the desired final proportion of NO in the combined gas mixture, i.e. a dosage, in other words in the combined gas resulting from the mixture of the flow of gas containing NO (i.e.) coming from the NO delivery apparatus with the flow of respiratory gas containing oxygen coming from the ventilator.
the dose adjustment means form part of an HMI (human-machine interface) or UGI (user graphics interface).
the dose adjustment means comprise one or more touch keys which can be actuated by the user and are displayed on a digital touch screen of the HMI, preferably of the type displaying in colour.
the set NO content is between 1 and 80 ppmv, typically between 5 and 40 ppmv.
it comprises storage means comprising a computer memory, for example a flash memory, RAM or the like.
it comprises control means comprising at least one (micro)processor, such as a microcontroller or the like.
the control means comprise one or more (micro)processors arranged on one or more electronic boards.
the control means comprise one or more (micro)processors which implement one or more algorithms, in particular one or more algorithms for operating or controlling valves, for processing flow rate or pressure measurements, etc.
the storage means are arranged on the electronic board.
it is supplied with electric power by one or more electric current sources, typically the mains supply (110/220 V) and/or one or more rechargeable batteries.

Furthermore, depending on the embodiment concerned, the anaesthesia ventilator may comprise one or more of the following features:
it incorporates one or more humidity and heat exchange filters making it possible to humidify the respiratory gas directly inside the ventilator, in other words upstream of the gas outlet of said anaesthesia ventilator.
it is configured to supply a flow of respiratory gas containing 02, preferably a respiratory gas containing at least 20 vol % of oxygen, typically at least 21 vol % of oxygen approximately.
the flow of respiratory gas containing O₂ leaving the anaesthesia ventilator contains water vapour, i.e. it is humidified.
preferably, the flow of respiratory gas containing O₂ leaving the anaesthesia ventilator has a temperature of between 15 and 30° C.
the respiratory gas containing O₂ is for example air or an oxygen/nitrogen (O₂/N₂) mixture.
it comprises a motorized blower (i.e. turbine, compressor or the like) delivering the respiratory gas, typically air or an oxygen/nitrogen mixture.
according to another embodiment, it comprises an internal gas circuit comprising one or more proportional valves for conveying the gas and controlling its supply, in particular its flow rate. This circuit is generally supplied with respiratory gas via one or more wall outlets supplied with gas from a network of pipes in a hospital establishment or building.
it comprises control means or a control device, such as one or more electronic control boards. Preferably, the control means of the medical ventilator operate or control the motorized blower or, depending on the circumstances, the proportional valves of the medical ventilator.
it is supplied with electric power by one or more electric current sources, typically the mains supply (110/220 V) and/or one or more rechargeable batteries.

Lastly, depending on the embodiment concerned, the NO container(s) supplying the installation of the invention may comprise one or more of the following features:
the (or each) NO container(s) contain an NO/N₂ mixture containing between 100 and 2000 ppmv of NO, the remainder being nitrogen, preferably an NO/N₂ mixture containing between 100 and 1500 ppmv, typically between 200 and 1000 ppmv.
when the NO containers are full, the NO/N₂ mixture therein is at a pressure of at least 150 bar, preferably at a pressure of between 10 and 250 bar (before gas is drawn off).
each NO container is or comprises one or more gas cylinders with a capacity of between 0.5 and 50 l (water equivalent).
each NO container is a pressurized gas cylinder.
each NO container comprises a cylindrical body made of steel or aluminium alloy and is equipped with a simple valve (without a regulator) or a valve with an integrated regulator or IRV, preferably an IRV.
the valve with which the/each container is equipped is protected by a protective cap, for example made of metal or polymer.

According to another aspect, the invention also relates to a method for therapeutic treatment of a person, i.e. a human patient (i.e. adult, child, adolescent or neonate), suffering from pulmonary hypertension and/or hypoxia, which cause pulmonary vasoconstrictions or the like, in particular in the case of surgery with gas anaesthesia, said method comprising administration by inhalation, to the person requiring it, of a gas mixture comprising from 1 to 80 ppmv of NO and at least 20 vol % of oxygen approximately, preferably at least 21 vol % of oxygen approximately, by means of a gas supply installation, as described above according to the invention, comprising an NO delivery apparatus for delivering NO and an anaesthesia ventilator in such a way as to treat (at least partially) said pulmonary hypertension and/or said hypoxia, in particular pulmonary hypertension caused by heart surgery with the patient being placed on extracorporeal blood circulation (ECC) and ventilatory anaesthesia of the patient.

### Definitions

In general, in the context of the invention:
“ppmv” means parts per million by volume.
“vol %” means percentage by volume.
“NO” denotes nitric oxide.
“NO₂” denotes nitrogen dioxide.
“N₂” denotes nitrogen.
“O₂” denotes oxygen.
the pressures are expressed in “bar absolute”, abbreviated to “bar”.
the terms “concentration”, “quantity”, “proportion”, “dose” and “content” are considered to be equivalent and interchangeable.
the terms “means of/to/for” are considered to be wholly equivalent to and interchangeable with the terms “device of/to/for”, for example the term “control means” may be replaced by “control device”, the term “valve means” may be replaced by “valve device”, the term “storage means” may be replaced by “storage device”, etc.
“pressure measurement” is to be understood as a pressure value (e.g. a numerical value) or a signal representative of such a pressure value, which reflects or corresponds to the gaseous pressure measured by a pressure sensor or the like.
“flow rate measurement” means a flow rate value (e.g. a numerical value) or one or more signals (pressure or flow rate) representative of such a flow rate value or making it possible to determine such a flow rate value, which reflects or corresponds to the gaseous flow rate measured by a sensor or the like.

### BRIEF DESCRIPTION OF THE VIEWS OF DRAWINGS

A clearer understanding of the invention will now be obtained from the following detailed description, given as a non-limiting example, with reference to the appended figures, in which:

FIG. 1 shows schematically an embodiment of an installation for administration of NO to a patient supplied by a conventional medical ventilator.

FIG. 2 shows schematically an embodiment of the connection of the flow rate measurement device, the NO injection device and the gas sampling module to the gas outlet of an anaesthesia ventilator according to the invention.

### DETAILED DESCRIPTION

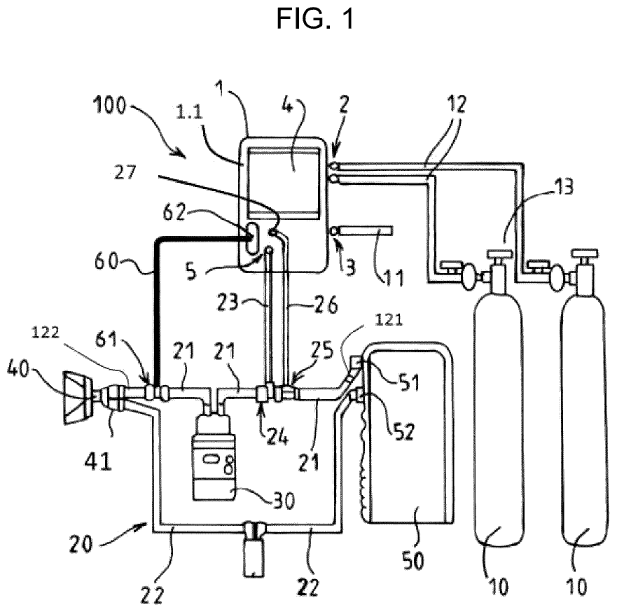

FIG. 1 shows schematically an embodiment of a gas administration installation 100 according to the invention, comprising an NO supply apparatus 1 which supplies a gas mixture based on nitric oxide (NO), and a conventional medical ventilator 50, in other words not an anaesthesia station or ventilator, which is adapted to supply a gas containing at least 20 vol % of oxygen, such as air or the like.

The installation 100 comprises NO sources 10, namely in this case two pressurized gas cylinders or containers each containing an NO/N₂ gas mixture, namely in this case an NO/N₂ gas mixture containing between 100 and 2000 ppmv of NO (remainder N₂), typically between 100 and 1000 ppmv of NO (remainder N₂), for example 450 or 800 ppmv of NO (remainder N₂), or any other suitable concentration, which supply an NO/N₂ mixture to the device or apparatus 1 for delivering or supplying NO, making it possible to monitor and control the supply of the NO/N₂ gas mixture.

The gas cylinders are each fluidically connected to the NO supply apparatus 1, via gas feed lines 12, such as flexible pipes or conduits or the like equipped with connectors, which may be furthermore equipped with devices for regulating and/or monitoring the gas pressure, such as a gas pressure-relieving valve 13, pressure gauges, etc.

The gas feed lines 12 are fluidically connected to gas inputs 2 of the NO delivery apparatus 1, which supply an internal gas circuit (not shown), used to convey the gas within the NO supply apparatus 1, i.e. in the outer casing or housing of the apparatus 1. Such an internal gas circuit comprises one or more conduit portions fluidically connected to the NO inlets 2 that are supplied with NO/N₂ mixture coming from the gas cylinders 10.

The gas circuit conventionally comprises solenoid valves or the like which are controlled by the control means of the apparatus 1 in order to control the flow of gas within the internal gas circuit, and other components, such as one or more pressure or flow sensors, for example an MFC (i.e. Mass Flow Controller).

The NO delivery apparatus 1 further comprises an oxygen inlet 3, which is fluidically connected, via an oxygen feed line 11, such as a flexible pipe or the like, to a source of oxygen (not shown), for example a pressurized oxygen cylinder or a hospital network, in other words an oxygen supply pipe which is provided in a hospital building. This makes it possible to feed the internal gas circuit 200 with oxygen when necessary.

In FIG. 1, the medical ventilator 50 is a conventional respiratory assistance apparatus, supplying a flow of oxygen-based respiratory gas, i.e. containing approximately at least 20 vol % of oxygen, preferably approximately at least 21 vol % of oxygen, such as air or an oxygen/nitrogen (N₂/O₂) mixture, but not making it possible to anaesthetize the patient.

The medical ventilator 50 and the NO supply apparatus 1 of the installation 100 are in fluidic communication with a respiratory circuit 20, also referred to as a patient circuit, which comprises an inspiratory branch 21 and an expiratory branch 22.

The inspiratory branch 21 serves to convey the gas flow to the respiratory interface 40 in order to supply the therapeutic gas flow to the patient, in other words a combined gas mixture containing the desired NO dosage.

The combined gas mixture to be administered to the patient is formed by mixing the oxygen-based flow (e.g. air or O₂/N₂ mixture) coming from the ventilator 50 and the NO-containing flow, i.e. the NO/N₂ gas mixture, delivered by the NO delivery apparatus 1.

For this purpose, the NO delivery apparatus 1 supplies or injects the NO/N₂ mixture into the inspiratory branch 21, via an injection conduit or line 23, which fluidically connects the internal gas circuit of the NO supply apparatus 1 to an injection device or module 24 arranged on the inspiratory branch 21. The injection device 24 is configured to mix the NO-containing gas with the flow of O₂-containing respiratory gas coming from the ventilator 50 and thus obtain the combined gas mixture containing NO and oxygen, i.e. the final gas mixture administered to the patient.

The injection device 24 comprises a first gas inlet supplied with a flow of O₂-containing respiratory gas from the medical ventilator 50, a second gas inlet supplied with NO-containing gas, i.e. gas coming from the NO delivery apparatus 1, and a gas outlet supplying the combined gas mixture containing NO and oxygen, obtained by mixing, within the injection device 24, the NO-containing gas with the flow of O₂-containing respiratory gas. The flow of NO/N₂ fed by the injection line 23 is then mixed, in an internal chamber or passage of the injection device 24, with the flow of gas based on oxygen (>20% O₂), e.g. air or an oxygen/nitrogen mixture, delivered by the ventilator 50 and conveyed by the inspiratory branch 21, so as to obtain the desired combined mixture, which is to be administered to the patient and contains essentially NO at the desired dosage, nitrogen (N₂) and oxygen (O₂), and possibly unavoidable impurities (e.g. argon, CO₂, NO₂, etc.), i.e. a final NO/N₂/O₂ gas mixture.

As can be seen in FIG. 1, when such an installation 100 is supplied by a conventional medical ventilator 50, the inspiratory branch 21 of the circuit 20 further comprises a gas humidifier 30 arranged downstream of the injection device 24. This makes it possible to humidify the combined gas flow, e.g. the NO/N₂/O₂ mixture, before it is administered by inhalation to the patient to be treated, by means of a respiratory interface 40, such as a tracheal intubation tube, a breathing mask or the like.

Moreover, the patient circuit 20 additionally comprises an expiratory branch 22 for recovering the gas exhaled by the patient. The inspiratory branch 21 (via a downstream end 122) and the expiratory branch 22 are fluidically connected to a junction piece 41, such as a Y piece, which is also connected to the respiratory interface 40 in order to ensure the exchanges of gas going to or coming from the patient's lungs.

In this conventional installation 100, the inspiratory branch 21 is fluidically connected, via its upstream end 121, to an outlet port 51 of the medical ventilator 50, such as a connector, coupling or the like, so as to recover and convey the oxygen-based gas, typically air or N₂/O₂ mixture, supplied by the medical ventilator 50, while the expiratory branch 22 is fluidically connected to an inlet port 52 of the medical ventilator 50, such as a connector, coupling or the like, so as to return to the medical ventilator 50 all or part of the flow of the gases exhaled by the patient (i.e. gases rich in CO₂).

In order to be able to measure the flow rate of gas delivered by the ventilator 50, such as air or N₂/O₂, circulating in the inspiratory branch 21, upstream of the injection device 24, there is a flow rate measurement device 25, typically a flow sensor, for example of hot wire, pressure differential or other type. The flow sensor 25 is connected to the NO delivery apparatus 1, via one or more flow rate measurement lines 26, which are connected to a connection port 27 of the apparatus 1.

Knowing the flow rate of respiratory gas makes it possible to control or regulate more efficiently the delivery of the flow of NO (i.e. N₂/O₂) by the NO delivery apparatus 1, in particular the flow rate of NO, since the flow rate measurements taken by the flow sensor 25 are returned to the (micro)processor control means of the NO delivery apparatus 1, typically a (micro)controller, which process these flow rate measurements to determine the flow of NO to be supplied, as a function of the desired dosage of NO and the proportion of NO in the flow of NO/N₂ coming from the gas cylinders 10.

Conventionally, the control means of the apparatus 1 (not shown as they are in the casing of the apparatus 1), such as a (micro)controller, comprise one or more electronic boards comprising one (or several) (micro)processors which implement one or more algorithms. They make it possible in particular to adjust or control the flow rate of NO-based gas by operating all or some of the (solenoid) valves, and also make it possible to perform calculations and/or to control or command all the electromechanical elements of the apparatus 1, such as the sensors, solenoid valves, displays, etc.

Usually, the NO supply apparatus 1 also comprises a graphical user interface (GUI) comprising a graphical display means 4, preferably a touch screen, i.e. with a touch panel, serving to display various pieces of information or data, icons, curves, alerts, etc., and also virtual selection keys and/or panes or windows, in particular for making choices, selections or for entering information, such as desired values (e.g. flow rate, dosage of NO, etc.), or any other information or data useful to the healthcare personnel. The display is preferably in colour, but it can also be in black and white.

The electrical power for the NO supply apparatus 1, in particular for the components requiring electrical current in order to operate, such as the control means, the graphical display means, etc., is provided conventionally by an electrical current source and/or electrical power supply means (not shown), for example a connection to the mains current (110/220 V), such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

In addition, the installation 100 also comprises a gas sampling line 60 which fluidically connects the inspiratory branch 21 to an inlet port 62 of the NO supply apparatus 1. The gas sampling line 60, such as a flexible pipe or the like, is connected to a sampling module 61 inserted in the inspiratory branch 21, between the humidifier 30 and the junction piece 41, i.e. the Y piece, typically in the immediate vicinity of the junction piece 41. The sampling module 61 and the gas sampling line 60 make it possible to take samples of combined gas and send them to the NO supply device 1 where they are analysed in an internal gas analyser (not shown) comprising NO₂, NO and O₂ sensors, typically electrochemical cells, electrically connected to the control means. This makes it possible to monitor the gas and check that it is compliant.

In particular, it should be verified that the composition of the combined gas complies with that of the desired NO/N₂/O₂ gas mixture to be administered to the patient, in particular in order to ensure that it does not contain excessive amounts of toxic NO₂ species, that its oxygen content is not hypoxic, that its NO₂ content is not too high, and that its NO content corresponds to the desired dosage, i.e. the dose of NO to be administered by inhalation, which is usually chosen by the healthcare personnel, i.e. physician or the like. The control means of the apparatus 1 process the signals coming from the sensors of the gas analyser and trigger alarms in the event that non-compliant proportions are determined. The NO, NO₂ and O₂ content values are preferentially displayed on the graphical display means 4 of the HMI.

In such an installation 1, the NO injection module 24 and the flow rate measurement device or flow sensor 25 are generally arranged in the inspiratory branch 21 upstream of the gas humidifier 30 and at a distance of at least 30 cm to 60 cm from the outlet of the ventilator 50, while the gas sampling line 23 is connected upstream of the Y piece 41, typically at around 15 to 40 cm therefrom.

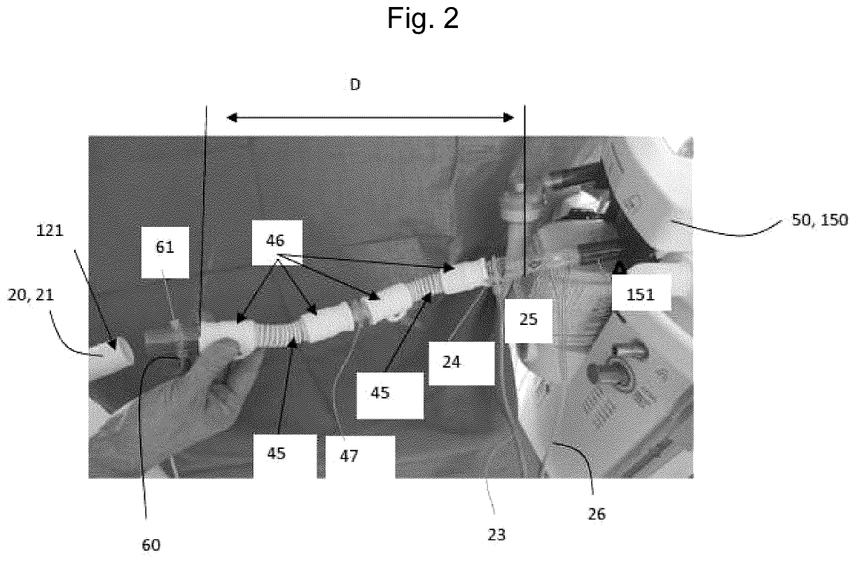

However, as explained above, when the ventilator 50 is, as shown in FIG. 2, an anaesthesia ventilator or station 150 such as those used during surgical operations, notably heart surgery with ECC, and when the patient circuit 20 is basic, in particular with a Y piece 41 sealed (i.e. non-removable) at the downstream end 122 of the inspiratory branch 21, it is not easy, or is even impossible, to connect, for the purposes of the surgery, the NO delivery apparatus 1 to the inspiratory branch 21 in order to supply NO to the patient and thus prevent or minimize pulmonary hypertension which may be caused by ECC in particular.

Therefore, the invention proposes a particular design or arrangement that is inserted between the patient circuit 20, typically the inspiratory branch 21, and the outlet 151 of the anaesthesia ventilator 150.

More specifically, as shown in FIG. 2, according to the invention, the flow rate measurement device or module 25 is connected directly to the gas outlet 151 of the anaesthesia ventilator 150 and no longer connected more than 30 cm to 60 cm away as in the case of a conventional ventilator 50 shown schematically in FIG. 1.

Furthermore, the gas sampling module 61 is itself arranged downstream of the flow rate measurement device or module 25 and at a distance D of at least 30 cm from the flow rate measurement device or module 25, being connected fluidically one to the other by means of one or more intermediate conduit portions 45, for example two intermediate conduit portions 45, as can be seen in FIG. 2, and tubular connectors 46.

It may also be necessary or desirable to use one or more additional fluidic connection elements 47 to connect for example two tubular connectors 46 to one another, as shown schematically in FIG. 2. Such tubular connectors 46 and additional fluidic connection elements 47 are conventional and are made for example of polymer.

The intermediate conduit portions 45 preferably comprise bendable or flexible pipes, for example made of polymer.

Moreover, the NO injection device 24 is connected between the flow rate measurement device or module 25 and the gas sampling module 61.

In the embodiment of FIG. 2, the NO injection device 24 is attached directly to the flow rate measurement device or module 25, for example one fitting in the other.

The intermediate conduit portions 45 thus make it possible to connect the outlet of the NO injection device 24 to the inlet of the gas sampling module 61.

The gas sampling module 61, which is arranged downstream of the NO injection device 24, is located at a distance D of between 30 cm and 80 cm, preferably 30 cm and 50 cm, from said NO injection device 24, which makes it possible to allow sufficient time for the mixture of the flow of NO/N₂ and the flow of respiratory gas (e.g. air or O₂/N₂) to become homogenized and for the gas samples taken by the gas sampling module 61 to be representative of the proportions of NO, O₂ and NO₂, as applicable, in the combined mixture obtained after mixing said flows.

As explained above, the flow rate measurement device 25 is connected to the NO delivery apparatus 1, i.e. to the connection port 27 of the apparatus 1, via the flow rate measurement line 26 serving to transmit the flow rate measurements taken by the flow rate measurement device 25. The flow rate measurement device 25 is preferentially a mass flow sensor and electrically connected to the NO delivery apparatus 1.

Likewise, the NO injection device 24 is connected, via an injection line or conduit 23, to the outlet port 5 of the NO delivery apparatus 1 in order to make it possible to convey and then inject the NO/N₂ mixture into the respiratory gas flow coming from the anaesthesia ventilator 150.

Similarly, the gas sampling module 61 is itself connected, via the gas sampling line 60, to the port 62 of the NO delivery apparatus 1 in order to bring to it the gas samples to be tested.

According to one embodiment, the injection device or module 24 comprises a module body comprising an internal gas passage, i.e. an internal volume or chamber, in fluidic communication with a first gas inlet supplied with a flow of O₂-containing respiratory gas, i.e. gas coming from the ventilator 150; a second gas inlet supplied with NO-containing gas coming from the NO delivery apparatus 1; and a gas outlet supplying the combined gas mixture containing NO and oxygen, obtained by mixing, within the internal gas passage of the injection device, the NO-containing gas (e.g. NO/N₂ mixture) with the flow of O₂-containing respiratory gas (e.g. air or O₂/N₂ mixture).

According to one embodiment, the flow rate measurement device 25 comprises a main body through which there passes an internal gas passage comprising an inlet hole through which the flow of respiratory gas containing O₂, i.e. coming from the ventilator 150, can enter the internal passage, and an outlet hole through which said flow of respiratory gas leaves the internal passage. It further comprises an electronic board acting as mass flow sensor borne by the main body and in fluidic communication with the internal passage so as to take gas flow rate measurements therein.

According to one embodiment, the gas sampling module 61 also comprises a main body through which there passes an internal gas passage comprising an inlet hole through which the flow of combined gas (i.e. NO/N₂/O₂ mixture) can enter the internal passage, and an outlet hole through which said flow of combined gas leaves the internal passage and is then conveyed by the inspiratory branch 21 of the patient circuit 20, as far as the patient. It further comprises a connector or the like for the fluidic connection of the gas sampling line 62 in such a way as to make it possible to take gas samples of the combined mixture within said internal passage, which are then brought by the gas sampling line 62 to the apparatus 1 where they are analysed.

In order to facilitate their fluidic connection, the flow rate measurement device 25, the NO injection device 24 and the gas sampling module 61 are preferentially equipped with tubular end pieces, also referred to as connectors, making it possible to connect them mechanically and fluidically in particular, depending on the case, to the outlet 151 of the anaesthesia ventilator 150, to the intermediate conduit portion or portions 45 and to the upstream end 121 of the inspiratory branch 21, in particular a connection by force-fitting, fitting together, or the like.

For example, the first gas inlet and the gas outlet of the injection device or module 24, the inlet hole and the outlet hole of the flow rate measurement device 25 and those of the gas sampling module 61 may be arranged in, i.e. borne by, such tubular end pieces.

In general, the flow rate measurement device 25, the NO injection device 24, the gas sampling module 61, said at least one intermediate conduit portion 45 and the tubular connectors 46 form an independent and removable connection assembly for connecting the NO delivery apparatus 1, which is connected fluidically between the inspiratory branch 21 of the patient circuit 20 and the gas outlet 151 of the anaesthesia ventilator 150, as shown schematically in FIG. 2, even when the patient circuit 20 is basic, for example formed of a simple gas conduit, and including when this circuit 20 comprises a Y piece that is sealed at the downstream end of the inspiratory branch 21, in other words not designed to be easily detached or removable from the inspiratory branch 21.

A gas administration installation 100 according to the invention may be used to administer nitric oxide (NO), i.e. the final NO/O₂/N₂ mixture obtained, by inhalation to persons, i.e. patients, suffering from acute pulmonary arterial hypertension, in particular to dilate their pulmonary vessels and increase their oxygenation by improving pulmonary gas exchange, in particular to treat pulmonary hypertension (PH) in heart surgery, in adults or children.

What is claimed is:

1. Installation (100) for supplying a gas mixture containing NO to a patient, comprising:
an NO delivery apparatus (1) configured to supply a flow of gas containing NO, and
a medical ventilator (50) for supplying a flow of respiratory gas containing O₂, to an inspiratory branch (21) of a respiratory circuit (20), said medical ventilator (50) being an anaesthesia ventilator (150) comprising a gas outlet (151),
an NO injection device (24), configured to inject the gas containing NO coming from the NO delivery apparatus (1) into the flow of respiratory gas coming from the medical ventilator (50),
a flow rate measurement device (25) configured to measure the flow rate of the flow of respiratory gas coming from the medical ventilator (50), the NO injection device (24) being arranged downstream of the flow rate measurement device (25), and
a gas sampling module (61) configured to sample, downstream of the NO injection device (24), some of the gas circulating in the inspiratory branch (21),
characterized in that the flow rate measurement device (25) is connected directly to the gas outlet (151) of the anaesthesia ventilator (150), and the gas sampling module (61) is arranged downstream of the NO injection device (24), at a distance (D) of at least 30 cm from said NO injection device (24).

2. Installation according to claim 1, characterized in that the NO injection device (24) is connected directly to the flow rate measurement device (25).

3. Installation according to claim 1, characterized in that the flow rate measurement device (25) is fluidically connected to the NO injection device (24) by means of at least one intermediate conduit portion (45).

4. Installation according to claim 3, characterized in that several intermediate conduit portions (45) fluidically connect the NO injection device (24) to the gas sampling module (61).

5. Installation according to claim 3, characterized in that tubular connectors (46) fluidically connect said at least one intermediate conduit portion (45) to the flow rate measurement device (25) and to the NO injection device (24), and/or several intermediate conduit portions (45) to one another.

6. Installation according to claim 1, characterized in that the gas sampling module (61) is arranged downstream of the NO injection device (24) and at a distance (D) of between 30 cm and 80 cm.

7. Installation according to claim 1, characterized in that the gas sampling module (61) is fluidically connected to the inspiratory branch (21) of the respiratory circuit (20).

8. Installation according to claim 4, characterized in that the gas sampling module (61) is fluidically connected to an upstream end (121) of the inspiratory branch (21).

9. Installation according to claim 1, characterized in that the respiratory circuit (20) comprises a junction piece (41) that is sealed at a downstream end (122) of the inspiratory branch (21), preferably the junction piece (41) is a Y piece.

10. Installation according to claim 3, characterized in that the flow rate measurement device (25), the NO injection device (24), the gas sampling module (61), said at least one intermediate conduit portion (45) and the tubular connectors (46) form an independent and removable connection assembly for connecting the NO delivery apparatus (1), configured to be fluidically connected between the inspiratory branch (21) of the patient circuit (20) and the gas outlet (151) of the anaesthesia ventilator (150).

11. Installation according to claim 1, characterized in that the gas sampling module (61) is arranged downstream of the NO injection device (24) and at a distance (D) of less than 60 cm.

12. Installation according to claim 1, characterized in that the gas sampling module (61) is arranged downstream of the NO injection device (24) and at a distance (D) of between 30 cm and 50 cm.

13. Installation according to claim 1, characterized in that the gas sampling module (61) is fluidically connected to an inlet of the inspiratory branch (21) of the respiratory circuit (20), said respiratory circuit (20) being a basic circuit comprising a sealed junction piece (41) located at a downstream end of the inspiratory branch (21).

14. Installation according to claim 1, characterized in that:
the NO injection device (24) is fluidically connected to the NO delivery apparatus (1) in such a way as to be supplied with gas containing NO by said NO delivery apparatus (1), and/or
the flow rate measurement device (25) comprises a flow sensor fluidically connected to the NO delivery apparatus (1) in such a way as to supply it with flow rate measurements, preferably the flow sensor is a mass flow sensor.

15. Installation according to claim 1, characterized in that the gas sampling module (61) is fluidically connected to the NO delivery apparatus (1) in such a way as to supply it with gas circulating in the inspiratory branch (21), downstream of the NO injection device (24).
